(19) United States
(12) Patent Application Publication
(10) Pub. No.: US 2004/0002546 A1
(43) Pub. Date: Jan. 1, 2004

(54) Methods for treating crohn's and other TNF associated diseases

(76) Inventor: Eric Altschuler, New York, NY (US)

Correspondence Address: FISH & RICHARDSON, PC, 12390 EL CAMINO REAL, SAN DIEGO, CA, 92130-2081, US

(21) Appl. No.: 10/244,037

(22) Filed: Sep. 14, 2002

Related U.S. Application Data
(60) Provisional application No. 60/322,892, filed on Sep. 15, 2001.

(51) Int. Cl. A61K 31/137

(52) U.S. Cl. 514/649

(57) ABSTRACT

A new method employing a known compound, bupropion hydrochloride (±)-1-(3-chlorophenyl)-2-[(1,1-dimethylethyl)amino]-1-propanone hydrochloride, and its analogues, in a new use for the treatment of TNF-related disorders is described.

### BACKGROUND OF THE INVENTION

This application claims priority to U.S. Provisional application number 60/322,892 filed Sep. 15, 2001.

1. Field of the Invention

The present invention generally concerns tumor necrosis factor (TNF) antagonists or TNF blockers for the treatment of autoimmnune diseases, inflammatory diseases, such as Crohn's, neurological disorders, trauma, injuries or compression, demyelinating neurological disorders, including multiple sclerosis; neurodegenerative diseases, including Alzheimer's disease; muscular disorders; and disorders of the optic nerve and retina (hereinafter “TNF-related Disorders”). More particularly, the small molecule TNF antagonists, TNF inhibitors or TNF blockers, are used for the treatment, prevention or amelioration of these TNF-related Disorders by modulating the action of TNF in the human body.

The present invention most particularly concerns a new method employing a known compound, bupropion hydrochloride (±)-1-(3-chlorophenyl) -2-[(1,1-dimethylethyl)amino]-1-propanone hydrochloride, and its analogues, in a new use for the treatment of these TNF-Related Disorders.

2. Description of Related Art

Inflammatory Diseases

A number of inflammatory agents, e.g. bacteria, viruses, injury, etc., can cause Inflammation. Inflammation is promoted at a cellular level by these agents binding to the cell surface. Once inflamed, a cell releases pro-inflammatory messenger proteins called cytokines. An inflammatory cascade is initiated when these inflamed cells cause neighboring cells to become inflamed.

Tumor necrosis factor (TNF), is a naturally occurring cytokine that is involved in the development and progression of many inflammatory, infectious and autoimmune diseases. In rheumatoid arthritis (RA), TNF-α, a major player in the pathology of joints, stimulates bone and cartilage absorption, facilitates inflammation and inhibits bone formation. The immune system is finely balanced either directly by the activities of pro-inflammatory and anti-inflammatory mediators or indirectly by their ability to regulate the production of other immunoregulatory molecules. At normal levels, the protein is essential for effective immune function. Overproduction of TNF as a result of age, genetic and other influences, however, contributes to the pathology of numerous diseases. High levels of TNF-α are present in patients with both RA and Crohn's disease.

When an immunomodulatory drug is administered, it blocks the inflammatory signal initiated at the cell surface. This results in a down-regulation of the inflammatory cascade. TNF-α overproduction can be suppressed in two ways: (1) by inactivating circulating levels of TNF-α with antibodies, or soluble receptors; and (2) by interfering with synthesis or release of TNF-α from producing cells.

TNF is formed by the cleavage of a precursor transmembrane protein, forming soluble molecules that aggregate to form trimolecular complexes. These complexes then bind to receptors found on a variety of cells. Binding produces an array of pro-inflammatory effects, including release of other pro-inflammatory cytokines, including interleukin (IL)-6, IL-8, and IL-1; release of matrix metalloproteinases; and upregulation of the expression of endothelial adhesion molecules, further amplifying the inflammatory and immune cascade by attracting leukocytes into extravascular tissues. TNF is now well established as key in the pathogenesis of inflammatory, autoimmune diseases such as rheumatoid arthritis (RA) and Crohn's Disease.

Other autoimmune diseases include, without limitation, diabetes melitus, multiple sclerosis, premature ovarian failure, scleroderm, Sjogren's disease, lupus, vilelego, alopecia (baldness), polyglandular failure, Grave's disease, hypothyroidism, polymyosititis, pempligus, colititis, autoimmune hepatitis, hypopituitarism, myocardititis, Addison's disease, autoimmune skin diseases, uveititis, pernicious anemia, and hypoparathyroidism.

Relatively recently commercially available, specific inhibitors of TNF, provide the possibility of therapeutic intervention in TNF-mediated diseases. Some therapeutic success in treating Crohn's Disease, RA and Psoriatic Arthritis has been demonstrated with, chimeric anti-TNF monoclonal antibodies (e.g., infliximab), and recombinant fusion proteins (e.g., etanercept). The latter consisting of two soluble TNF receptors joined by the Fc fragment of a human IgG1 molecule. Other specific anti-TNF agents under development, including D2E7 (a human anti-TNF mAb), CDP 571 (a chimeric, but 95% humanized, anti-TNF mAb) and a pegylated soluble TNF type 1 receptor are also somewhat successful. Thalidomide and its functional analogues “SelCIDs” and “ImiDs” are anti-TNF agents recently used. None of these modalities has been very successful. There is a need for small, effective molecular TNF that would be non-toxic, stable, non-immunogenic and capable of passing the blood-brain barrier. Other innovative therapies might include gene therapy and the development of selective TNF production inhibitors, such as the TNF-converting enzyme.

### Neurological Disorders

As with other organ systems, TNF has been shown to have a key role in the central nervous system. TNF-α, a protein previously considered to be only a component in the immune system, actually plays a key role in regulating neurotransmission in the central nervous system as well. It has long been recognized as being a key player in controlling cell death, but this new finding offers new insights into how cells interact within the human nervous system. This new role of TNF-α may provide researchers with possible new approaches to treating illnesses such as dementia, Alzheimer's disease, stroke, epilepsy and spinal cord injury.

Scientists have long believed that neurons were the really important cells in the nervous system because they control transmittal of signals within the entire CNS. Glial cells, comprising astrocytes, oligodendrocytes and microglia, were given credit only in a supportive role for the neurons, providing oxygen and nutrients to the neurons, shielding neurons from each other, and basically cleaning up dead neurons. New research, however, points to a much greater role for the glial cells—they can manufacture and release TNF-α into the CNS. The TNF-α has been shown to be able to regulate the expression of certain neurotransmitter receptors on the surface of neurons.

The binding of glutamate molecules from the fluid surrounding the cell to these receptors initiates signal production. When the glutamate and receptor meet, a nerve impulse, or signal, is produced. The more receptors present on the neuron surface, the more signals are produced. Normally, TNF-α is released as part of the inflammatory process following an injury to the cells. When TNF-α and glutamate are both present, cell-signaling activity seems to increase.

Because there is glutamate and TNF-α present in the spinal cord after injury, then perhaps TNF-α is actually enhancing the killing effect of the normal neurotransmitter. When nerve cells were exposed first to glutamate and then to TNF-α separately, neither had an impact on the normal killing rate. However, when the cells were exposed to even small amounts of both compounds, the killing effect increased by 120 percent.

Demyelinating disease neurological disorders (e.g. multiple sclerosis), immune disease, inflammation, trauma or compression, occur in different clinical forms, depending upon the anatomic site and the cause and natural history of the physiological problem. In Alzheimer's disease, for example, the brain undergoes serious neurodegeneration of unknown etiology. The fact that they can cause permanent neurological damage, that the damage can occur rapidly and be irreversible, and that current treatment of these conditions is unsatisfactory, is a common thread in all of these disorders.

Neurological conditions associated with TNF-α overproduction include, but are not limited to, acute spinal cord trauma, spinal cord compression, spinal cord hematoma, cord contusion, nerve compression, e.g., a herniated disc causing sciatic nerve compression, neuropathy, and pain. Also included are: cervical disc herniation, causing nerve compression in the neck; acute or chronic spinal cord compression from cancer metastasis; autoimmune disease of the nervous system; and demyelinating diseases, the most common condition being multiple sclerosis as stated hereinabove.

Treatment modalities employing steroid drugs such as cortisone to treat many of the aforementioned neurological problems and conditions are particularly hazardous because they are used either at high dosage, with a corresponding increasing risk of side effects, or because they are used chronically. Moreover, steroids are only partially effective or completely ineffective.

The ability of the body to repair injury to the nervous system is limited. The devastating nature of these diseases and the lack of effective therapy underscore the urgent need for early therapy to prevent or limit neuronal death. Therapies directed against TNF to dramatically limit inflammation by interrupting the inflammatory cascade at a fundamental level should be ideally suited. However, only very limited success has been achieved.

Therefore, there is a need for new TNF inhibitors therapeutically effective for a wide variety of neurological and related disorders. As stated hereinabove, these disorders are diverse. These further include Guillain Barre syndrome, and myasthenia gravis; degenerative disorders of the nervous system, including Parkinson's disease and Huntington's disease; disorders of related systems of the retina and of muscle, including optic neuritis, macular degeneration, diabetic retinopathy, dermatomyositis, amyotrophic lateral sclerosis, and muscular dystrophy; and injuries to the nervous system, including traumatic brain injury, acute spinal cord injury, and stroke.

There remains a need for a new pharmacologic treatment of these aforementioned physiological problems of the nervous system associated with autoimmune disease, demyelinating diseases, neurodegenerative diseases, trauma, injuries and compression with the pharmacological use of new TNF antagonists or TNF blockers that will be beneficial for the large number of patients afflicted. Drugs such as etanercept, infliximab, pegylated soluble TNF Receptor Type I (PEGs TNF-R1), other agents containing soluble TNF receptors, CDP571 (a humanized monoclonal anti-TNF-α antibodies), thalidomide, phosphodiesterase 4 (IV) inhibitor thalidomide analogues and other phosphodiesterase IV inhibitors are generally unsatisfactory because they provide either no remission, or only partial alleviation of suffering.

The current treatments with injectable proteins have risks and limitations. An orally active molecule that reduces TNF-/IL-1 synthesis could either replace the use of the injectables or provide better disease control when used in conjunction with other therapies. Additionally, several of these TNF agents will not cross the blood-brain barrier. It is painfully obvious that a small, effective, non-immunogenic molecule is needed.

Pharmacologic chemical substances, compounds and agents which are used for the treatment of neurological disorders, trauma, injuries and compression having various organic structures and metabolic functions have been disclosed in the prior art. For example, U.S. Pat. Nos. 5,756,482 and 5,574,022 to Roberts et al disclose methods of attenuating physical damage to the nervous system and to the spinal cord after injury using steroid hormones or steroid precursors such as pregnenolone, and pregnenolone sulfate in conjunction with a non-steroidal anti-inflammatory substance such as indomethacin. These patents do not teach the use of a TNF antagonist or TNF blocker for the suppression and inhibition of the action of TNF in the human body to treat TNF-related disorders, as in the present invention.

U.S. Pat. No. 5,605,690 to Jacobs discloses a method for treating TNF-dependent inflammatory diseases such as arthritis by administering to a human a TNF antagonist, such as soluble human TNFR, a peptide. This patent does not teach the use of a TNF antagonist or TNF blocker, as in the present invention.

U.S. Pat. No. 5,656,272 to Le et. al discloses methods of treating TNF-mediated Crohn's disease using chimeric anti-TNF antibodies. This prior art patent does not teach the use of a TNF antagonist or TNF blocker, as in the present invention.

U.S. Pat. No. 5,650,396 discloses a method of treating multiple sclerosis by blocking and inhibiting the action of TNF in a patient. This prior art patent does not teach the use of the TNF antagonist in the present invention.

U.S. Pat. No. 6,428,787, issued to Tobinick on Aug. 6, 2002, disclosed a myriad of molecular compounds purportedly effective in treating inflammatory diseases by inhibiting TNF-α, but there is no evidence that these are in any way effective in humans.

None of the prior art patents disclose or teach the use of TNF antagonists blockers of the present invention for inhibition TNF production in a human to treat TNF-related disorders, in which the TNF antagonist affords the patient complete remission or opportunity to heal, slows disease progression, prevents neurological damage, or otherwise improves the patient's life.

Accordingly, it is an object of the present invention to provide TNF antagonists for a new pharmacologic treatment of TNF-related disorders, such that the use of these TNT antagonists will result in significant amelioration of these conditions.

Another object of the present invention is to provide a TNF antagonist for providing suppression and inhibition of the action of TNF to treat TNF-related disorders.

Another object of the present invention is to provide a TNF antagonist that reduces inflammation to a patient by inhibiting the action of TNF for the immediate, short term and long term, such that this reduction in inflammation will produce clinical improvement in the patient to heal, slow disease progression, prevent neurological damage, or otherwise improves the patient's life.

Another object of the present invention is to provide TNF antagonists that can offer acute and chronic treatment for neurological conditions caused by neurological trauma, compression, injury or disease; such conditions including acute spinal cord or brain injury, herniated nucleus pulposus, spinal cord compression due to metastatic cancer, primary or metastatic brain tumors, chronic pain syndromes due to metastatic tumor, increased intracranial pressure, demyelinating diseases such as multiple sclerosis, neurodegenerative diseases such as Alzheimer's disease, inflammatory CNS disease, such as subacute sclerosing panencephalitis, and other related neurological disorders and diseases.

Another object of the present invention is to provide a TNF antagonist that can offer acute and chronic treatment regimens for neurological and related diseases. Examples of diseases in these categories include but are not limited to diseases of the central and peripheral nervous system such as Parkinson's disease, Bell's palsy, Guillain-Barre syndrome.

Another object of the present invention is to provide a TNF antagonist that can offer acute and chronic treatment for retinal and neuro-ophthalmic diseases. Examples of diseases in these categories include but are not limited to optic neuritis, macular degeneration and diabetic retinopathy.

Another object of the present invention is to provide a TNF antagonist that can offer acute and chronic treatment for muscular diseases and diseases of the neuromuscular junction. Examples of diseases in these categories include but are not limited to dermatomyositis, amyotrophic lateral sclerosis and muscular dystrophy.

Another object of the present invention is to provide a TNF antagonist that can offer acute and chronic treatment regimens for degenerative neurological disorders and neurologic disorders of uncertain etiology. Examples of diseases in these categories include but are not limited to Alzheimer's disease, Huntington's disease and Creutzfeld-Jakob disease.

Another object of the present invention is to provide a TNF antagonist that can offer acute and chronic treatment regimens for neurologic injuries. Examples of diseases in these categories include but are not limited to acute spinal cord injury, acute brain injury and stroke.

Another object of the present invention is to provide a TNF antagonist that can offer acute and chronic treatment regimens for inflammatory and autoimmune disorders of the nervous system, examples being subacute sclerosing panencephalitis and myasthenia gravis.

Another object of the present invention is to provide a TNF antagonist that can offer acute and chronic treatment that is not hazardous to a patient.

Yet another object of the present invention is to provide a TNF antagonist that can offer acute and chronic treatment that is not prohibitively expensive.

Other objects will become apparent to a person skilled in the art upon contemplating this invention as described hereinbelow, such objects being within the spirit of this invention.

### SUMMARY OF THE INVENTION

In accordance with the above-stated objects, this invention contemplates a method for inhibiting the action of TNF for treating neurological conditions in a human by administering a TNF antagonist, bupropion hydrochloride or its therapeutically effective analogue, for reducing the inflammation of neuronal tissue or the neuromuscular junction, or for modulating the immune response affecting neuronal tissue or the neuromuscular junction.

The present invention further provides a method for inhibiting the action of TNF for treating conditions of the optic nerve or retina in a human by administering a TNF antagonist, bupropion hydrochloride or its therapeutically effective analogue, for reducing the inflammation of the optic nerve or retina, or for modulating the immune response affecting the optic nerve or retina.

The present invention also provides a method for inhibiting the action of TNF for treating muscular diseases in a human by administering a TNF antagonist, bupropion hydrochloride or its therapeutically effective analogue, for reducing the inflammation of muscle of a human, or for modulating the immune response affecting the muscle. The TNF antagonist is selected from the group consisting of buproprion and its analogues.

In the step of administering the TNF antagonist to a human, the TNF antagonist is performed through any of the following routes including subcutaneous, intravenous, intrathecal, intramuscular, intranasal oral, or transepidermal.

In another aspect, the invention relates to a pharmaceutical composition comprising, as an active component, a compound of bupropion hydrochloride or its therapeutically effective analogue, together with a pharmaceutically acceptable excipient or carrier. The term “pharmaceutically acceptable” is intended to indicate that the excipient or carrier included in the composition is compatible with the other ingredients and not toxic or otherwise deleterious to a patient to whom the composition is administered.

Pharmaceutical compositions of the invention may be in unit dosage form such as tablets, pills, capsules, powders, granules, elixirs, syrups, emulsions, ampoules, suppositories or parenteral solutions or suspensions for oral, parenteral, opthalmic, transdermal, intra-articular, topical, pulmonal, nasal, buccal or rectal administration or in any other manner appropriate for the formulation of anti-inflammatory compounds and in accordance with accepted practices such as those disclosed in Remington: The Science and Practice of Pharmacy. 19.sup.th Ed., Mack Publishing Company, 1995. The term “unit dosage” is intended to indicate a unitary, i.e. a single dose which is capable of being administered to a patient, and which may be readily handled and packed, remaining as a physically and chemically stable unit dose comprising either the active component as such or a mixture of it with solid or liquid pharmaceutical excipients or carriers. In the composition of the invention, the active component may be present in an amount of from about 0.1-100% by weight of the composition.

For oral administration in the form of a tablet or capsule bupropion hydrochloride or its therapeutically effective analogue may suitably be combined with an oral non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methylcellulose, agar, bentonite, xanthan gum or the like.

For the preparation of solid compositions such as tablets, the active compound of bupropion hydrochloride or its therapeutically effective analogue is mixed with one or more excipients, such as the ones described above, and other pharmaceutical diluents such as water to make a solid preformulation composition containing a homogenous mixture. The term “homogenous” is understood to mean that the compound of bupropion hydrochloride or its therapeutically effective analogue is dispersed evenly throughout the composition so that the composition may readily be subdivided into equally effective unit dosage forms such as tablets or capsules.

Liquid formulations for either oral or parenteral administration of the compound of the invention include, for example, aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, for example, intramuscular, intraperitoneal, subcutaneous or intravenous injection or infusion, the pharmaceutical composition preferably comprises a compound of bupropion hydrochloride or its therapeutically effective analogue dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances, in particular antiproliferative agents. The composition may be sterilized by, for instance, filtration through a bacteria-retaining filter, addition of a sterilizing agent to the composition, irradiation of the composition, or heating the composition.

Alternatively, the compound of the invention may be provided as a sterile, solid preparation, for example, a freeze-dried powder that is dissolved in sterile solvent immediately prior to use.

The composition intended for parenteral administration may additionally comprise conventional additives such as stabilizers, buffers or preservatives, for example, antioxidants such as methylhydroxybenzoate or the like. Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient, bupropion hydrochloride or its therapeutically effective analogue, which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. For topical administration, the active component preferably comprises from 1% to 20% by weight of the composition, but the active ingredient may comprise as much as 50% w/w.

Compositions suitable for administration of bupropion hydrochloride or its therapeutically effective analogue to the nasal or buccal cavity or for inhalation include powder, self-propelling and spray formulations, such as aerosols and atomizers. Compositions suitable for nasal or buccal administration may comprise 0.1% to 20% w/w. for example about 2% w/w of active ingredient.

The composition may additionally comprise one or more other active components conventionally used in the treatment of various inflammatory diseases and conditions. Examples of such additional active components may be selected from the group consisting of glucocorticoids, vitamin D and vitamin D analogues, antihistamines, platelet activating factor (PAF) antagonists, anticholinergic agents, methylxanthines, .beta.-adrenergic agents, COX-2 inhibitors, salicylates, infomethacin, flufenamate, naproxen, timegadine, gold salts, penicillamine, serum cholesterol lowering agents, retinoids, zinc salts and salicylazosulfapyridine.

A suitable dosage of the compound of the invention will depend, inter alia, on the particular compound selected for the treatment, the route of administration, the age and condition of the patient, the severity of the disease to be treated and other factors well known to the practicing physician. The compound may be administered either orally or parenterally according to different dosing schedules, for example, daily or with weekly intervals. In general, a single dose will be in the range from 0.01 to 500 mg/kg body weight, such as from 0.1 to 150 mg/kg body weight. The compound may be administered as a bolus or in divided doses two or more times a day.

The invention is further described in the following general procedures, preparations and examples that are not in any way intended to limit the scope of the invention as claimed.

### DESCRIPTION OF THE PREFERRED EMBODIMENTS

Cytokines and growth factors, the cellular mediators of the immune system, have profound effects on disease processes. The immune system is finely balanced either directly by the activities of pro-inflammatory and anti-inflammatory mediators or indirectly by their ability to regulate the production of other immunoregulatory molecules. Unregulated activities of these mediators can lead to the development of serious inflammatory and other diseases. Enhanced tumor necrosis factor-(TNF-) and interleukin-1 (IL-1) levels are associated with the development of rheumatoid arthritis, psoriatic arthritis and inflammatory bowel disease.

Examples below are illustrative only and are not meant to be restrictive. A skilled worker in the field will realize immediately that variations are abundant.

### EXAMPLE 1

### Crohn's Disease

a. Manifestation

Crohn's disease is a chronic and serious inflammatory disease of the gastrointestinal that eventually breaks down the intestinal wall. It afflicts more than 500,000 Americans, most of them under age 30. People with Crohn's disease may experience a number of symptoms including diarrhea, abdominal cramps and pain, fever, rectal bleeding, loss of appetite, and weight loss. The range and frequency of these symptoms varies. About half of all Crohn's patients at some point require surgery to remove intestinal obstructions or repair holes in the bowel. Crohn's disease is also sometimes referred to as ileitis, regional enteritis or colitis. Although Crohn's disease is the general term used to describe the condition, doctors use these specific terms to differentiate the portion of the bowel that is involved.

### Complications of Crohn's Disease

The complications that patients of Crohn's disease depends upon the location and severity of the disease.

a. Serious Complications

The swelling and scarring associated with Crohn's disease can obstruct the intestine. When this occurs, a patient may experience painful cramps, or vomiting due to food that is not able to bypass the obstruction. Patients who experience an obstructive episode are usually hospitalized and given intravenous fluids and salts to compensate for fluid losses. Ulcers caused by Crohn's disease may channel through the gut wall to adjacent organs. Fistulas tracking into the abdominal cavity may produce infections and form abscesses. And, if a fistula fails to drain, abscesses may occur in the pelvis or the perineum. These may require surgery, and are treated usually treated with antibiotics to reduce the risk of further infection.

Rarely, penetrating ulcers tunnel through the layers of the gut into the abdominal cavity. This free perforation manifests in sudden abdominal pain, shock and excessive tenderness. The resulting peritonitis requires emergency surgery to seal the leak and clean the abdominal cavity to stop further infection. In other rare cases, a bleeding ulcer may burrow through the gut wall and shear an artery, producing life-threatening hemorrhaging and requiring emergency surgery.

b. Chronic Complications

Anemia, malnutrition and growth retardation are among chronic complications of Crohn's. Anemia may be the result of chronic blood loss, bone marrow depression, or failure to absorb iron and folic acid, all important in stimulating the production of red blood cells. Certain treatment medications, such as sulfasalazine, may exacerbate anemia, requiring diet supplementation. Loss of protein, tissue breakdown, poor diet and failure to absorb nutrients, may result in nutritional deficiencies and weight loss. About one-third of children with Crohn's experience growth retardation. Treatment

The etiology of Crohn's disease has not yet been discovered. Presently, treatment goals are to suppress inflammation, facilitate healing; and relieve the symptoms. It is a chronic inflammatory disease of the bowel with overactivity of the immune cells within the bowel, such as macrophages, lymphocytes and neutrophils that reside in the wall just below the lining or mucosal surface. If the intestinal mucosa is breached by bacteria, such as E.coli or Salmonella, these cells isolate and kill the bacteria. Under normal circumstances, the bowel returns to normal.

### Conventional Treatments

Sulfasalazine and mesalamine have shown efficacy in treating patients with mild to moderately active Crohn's. Some of these medications are available in enema or suppository form to target specific levels of the bowel. Antibiotics are used to control symptoms involving the colon, distal small bowel and perianal region. The antibiotics decrease the concentration of the bacteria residing in the bowel diminishing concentration of the breakdown products released when they die. These breakdown products may contribute to the inflammation associated with Crohn's disease. Side effects of the treatments include nausea, a metallic taste and peripheral neuropathy characterized as numbness in the hands and feet, resolving when the medication is discontinued. These treatments are expensive.

Corticosteroids are potent immunosuppressive medications used to treat inflammation in active Crohn's disease. Preparations available for oral, rectal and intravenous administration, work quickly and are fairly inexpensive. Unfortunately, when used long term they have many undesirable side effects, including adrenal gland suppression, hypertension diabetes, osteoporosis cataracts and hip fracture. Entocort EC capsules, formulated to release budesonide once they reach the intestine, is purported to have fewer side effects than other steroids, such as prednisone. About half of patients taking Entocort EC experienced improvement in their symptoms after two months of therapy.

Azathioprine, 6-mercaptopurine and Methotrexate are immunosuppressive medications used when inflammation cannot be controlled with corticosteroids. As with all immunosuppressive medications, these drugs have substantial side effects.

New Anti TNF-Therapy

Infliximab, a monoclonal antibody, was released by the U.S. Food and Drug Administration in August 1998 to treat patients with moderate to severe Crohn's disease that is resistant to management with the immunosuppressors and medications above. Specifically acting against TNF-α, it has been studied in two randomized-controlled trials involving approximately 100 patients with Crohn's disease. Four weeks after receiving the medication, 50 to 81 percent of the patients showed signs of improvement and 33 percent went into remission, while 17 percent in the placebo group improved and 4 percent entered remission. In the second study, patients with active fistulas received infliximab or placebo on three occasions, two weeks apart. There was a 50 percent reduction in fistula drainage in nearly two-thirds of patients who received infliximab, and over half of the patients' fistulas closed. Although significant, the results are not particularly appreciated, especially by the great number of patients not responding.

The anti-TNF-α therapy is contraindicated in patients with an active infection and in patients with a history of malignancy because of the role that TNF-plays in combating the development and spread of cancer. Allergy to the medication and pregnancy are other contraindications. Patients with marked narrowing of the bowel should not receive this medication, because rapid healing may lead to scar tissue formation, resulting in complete bowel obstruction. Finally, anti TNF-α therapy is expensive, and no data is available on its long term use.

When the Food and Drug Administration gave early approval to the new drug Remicaid a little more than a year ago, it offered hope particularly to patients with the worst type of Crohn's—those with ulcers, or fistulas, that tunnel through the bowel wall into nearby organs or through the surface of the skin. In one study, half of the fistulas closed in 68 percent of patients taking Remicaid. In another, 48 percent of patients showed complete remission.

Yet Remicaid has big risks. Such immunosuppression has been linked to lymphomas and other cancers. The FDA, ending the Remicaid trials after only three years in order to get the drug to desperately ill patients, closed the window on evaluating the drug's long-term effects.

### Antisense Therapy

Isis Pharmaceuticals announced that it has initiated Phase I clinical studies of ISIS 104838, a novel antisense inhibitor of TNT-α employing second-generation antisense chemistry, to treat inflammatory and autoimmune diseases such as rheumatoid arthritis (RA) and Crohn's. It will investigate the safety and efficacy of the drug administered intravenously (IV) and subcutaneously. Oral formulations are being developed in parallel.

### Small Molecular Inhibitors

Celgene has identified this route as being accessible through its two classes of small molecule, orally available agents: “SelCIDS” and “ImiDs”. These agents use different biochemical mechanisms to target underlying TNF-α overproduction without affecting general immune system function. However the results are not encouraging.

### Treatment According to this Invention

Some patients with Crohn's disease (CD) are not controlled by current treatments. Also, multiple toxicity issues are associated with taking medicines such as azathioprine, methotrexate, and steroids over time (Sartor R B. New therapeutic approaches to Crohn;s disease. N Engl J Med 2000;342:1664-1666). Inventors herein point out that the literature strongly supports the notion that monoaminergic and dopaminergic treatments can lower levels of TNF-α via increased intracellular adenosine 3′,5′-cyclic monophosphate (cAMP), and describe two cases in which bupropion hydrochloride has produced near complete and long-lasting (over 2 years) remissions of CD without significant side effects or toxicities.

In CD, TNF is increased, and TNF is thought to play a central role in CD pathogenesis (Papadakis K A, Targan S R. Role of cytokines in the pathogenesis of inflammatory bowel disease. Annu Rev Med 2000;51:289-298).

A monoclonal anti-TNF antibody has been found of some efficacy in CD, for example, in closing fistulas (Present D H, Rutgeerts P, Targan S. Hanauer S B, Mayer L, van Hogezand R A, Podolsky D K, Sands B E, Braakman T, De Woody K L, Schaible T F, van Deventer S J. Infliximab for the treatment of fistulas in patients with Crohn's disease. N Engl J Med 1999;340:1398-1405). However, the antibody is expensive, it must be given intravenously, and although not common, there is potential for severe side effects. Also, long-term effects of repeated treatments with anti-TNF antibody are unknown. In CD studies, even in patients who benefit from a given medicine, frequently, enough CD activity remains to significantly lower quality of life even though the Crohn's Disease Activity Index (CDAI) (Best W R, Becktel J M, Singleton J W. Rederived values of the eight co-efficients of the Crohn's Disease Activity Index (CDAI). Gastroenterology 1979,77:843-846) is under the somewhat arbitrary cutoff of 150 used to define remission. Clearly, easing of disease burden is of great value, but remission of all signs and symptoms of disease remains our goal.

Agonist binding to the β-adrenergic receptor increases intracellular cAMP (Talmadge J, Scott R, Castelli P, Newman-Tarr T, Lee J. Molecular pharmacology of the β-adrenergic receptor on THP-1 cells. Int J Immunopharmacol 1993;15:219-228). cAMP is believed to be a key intracellular regulator of TNF, and increases of cAMP from any origin have been shown to decrease TNF in a variety of in vitro and in vivo situations in animals and humans (Prabhakar U, Lipshutz D, Bartus J O, Slivjak M J, Smith E F 3rd, Lee J C, Esser K M. Characterization of cAMP dependent inhibition of LPS induced TNF production by rolipram, a specific phosphodiesterase IV inhibitor. Int J Immunopharmacol 1994.16:805-816; Guirao X, Kumar A, Katz J, Smith M, Lin E, Keogh C, Calvano S E, Lowry S F. Catecholamines increase monocyte TNF receptors and inhibit TNF through-2 adrenoreceptor activation. Am J Physiol 1997;273:E1203-1208; Sommer N. Loschmann P A, Northoff G H, et al. The antidepressant rolipram suppresses cytokine production and prevents autoimmune encephalitis. Nat Med 1995;1:244-248). Phosphodiesterases mediate a step in CAMP catabolism. Phosphodiesterase inhibitors increase cAMP levels and lower TNF levels. The phosphodiesterase inhibitor rolipram has been shown in different experimental models to not only lower TNF levels, but also be effective in mitigating disease severity in several animal models of human inflammatory/autoimmune disease (e.g., Sommer et al.). Recently, the β-adrenergic agonist isoproteronol has been found to lower TNF levels when used into humans (Goebel M U, Mills P J, Irwin M R, Ziegler M G. Interleukin-6 and tumor necrosis factor-alpha production after acute psychological stress, exercise, and infused isoproterenol: differential effects and pathways. Psychosom Med 2000;62:591-598).

Similarly, Inventors suggest that cases of CD remission seen after phenelzine treatment of depression (Kast R E. Crohn's disease remission with phenelzine. Gastroenterology 1998;115:1034-1035) may be driven by increased cAMP-associated TNF decreases. Consistent with this notion, in a fascinating recent abstract it has been found that the levels of norepinephine were lower in gut mucosa in CD patients than in healthy controls or ulcerative colitis (UC) patients, and dopamine levels were lower in gut mucosa in CD and UC patients than in controls (Magro F, Vieira-Coelho M A, Fraga S, Serrao M P, Tavarela-Veloso F, Tome-Ribeiro, Soares-da-Silva P. Impaired synthesis or cellular storage of norepinephrine, dopamine and 5-hydroxytryptamine in inflammatory bowel disease (abstr). Am J Gastroenterol 2000;95:2556). Also we note that, amazingly, an MAO-I ( Leib J. Remission of rheumatoid arthritis and other disorders of immunity in patients taking monoamine oxidase inhibitors. Int J Immunopharmacol 1983;5:353-357) has been reported to induce remission in rheumatoid arthritis, and we have suggested that increased monoamine-mediated TNF decrease is the mechanism behind this observation as well (Altschuler EL. Monoamine oxidase inhibitors in rheumatoid arthritis-anti-tumor necrosis factor? Int J Immunopharmacol 2000;22:1007-1008).

Given the long-term experience with and safety of phenelzine, further study of phenelzine in CD might be considered. However, phenelzine carries some risks: hypertensive crisis associated with dietary restriction breaches can be serious, and fatal hypertensive crises are seen with multiple medicine incompatibilities (e.g., meperidine). Other medicines that can increase monoaminergic tone without these risks could be considered. Bupropion hydrochloride is an antidepressant without the risks of MAOIs. The mechanism of bupropion's antidepressant action is unclear, but might involve weak inhibition of catecholamine re-uptake (Sanchez C, Hyttel J. Comparison of the effects of antidepressants and their metabolites on reuptake of biogenic amines and on receptor binding. Cell Mol Neurobiol 1999;19:467-489). Bupropion seems to be unique in that it is the only modem antidepressant that does not cause compensatory β-adrenergic receptor down-regulation (Garrcha G, Smokcum R W, Stephenson J D, et al. Effect of some atypical antidepressants on β-adrenoreceptor binding and adenylate cyclase activity in the rat forebrain. Eur J Pharmacol 1985;108:1-7).

A 44-year-old woman presented in May of 1999 for treatment of an episode of major depression, superimposed on a chronic mild depressed state (dysthymia). She had a 10-year history of active CD, radiographically limited to the colon, with a history of 1 resection of a very small section of large bowel. She was not usually pain-free during the week, though usually did have several pain-free hours in a given day. Overt blood was noted in the stool frequently. Inadvertent loss of stool several times a month required daily use of adult protective pads. She was taking fluoxetine 40 mg every day for depression, and mesalamine 500 mg twice a day. CDAI was 202. She had a history of approximately 1 CD flare per year requiring steroids.

After careful review of treatment options, she was started on bupropion (fluoxetine was stopped) with up-titration to 150 mg twice a day. Her abdominal complaints eased over the next months, and the major depression remitted. The baseline dysthymia remained. To better treat the dysthymia, bupropion was raised to 150 mg three times daily with a further lowering of her CDAI. The mesalamine was tapered off. Her CDAI is currently approximately zero. She has one well-formed bowel movement daily and has been without abdominal symptoms or encopresis for 19 months now. She has had no CD flares since starting bupropion. Twice in the last year, she stopped bupropion on her own accord because she felt completely well and felt she didn't need bupropion anymore. Abdominal cramping, and blood per rectum recurred on both occasions, but remitted within weeks of restarting bupropion.

After seeing this patient's response to bupropion, Inventors inquired with colleagues of whether they knew of similar responses in CD patients. They were referred to the case of a 45-year-old man with a 20-year history of Crohn's enteritis with multiple surgeries, including 4 small bowel resections. In April of 1999, even though he was on azathioprine, the patient had a CD flare requiring bowel resection. After resolution of the flare, a retrospective history shows CDAI of approximately 275. The patient was taking fluoxetine to help with pain control. Because this was not effective and also because the patient was a smoker, it was suggested to the patient that he consider switching from fluoxetine to bupropion, and he did so. Even though he was not able to stop smoking, the patient felt better on bupropion so he decided to continue this medicine with his family doctor's consent. Currently, the patient is taking bupropion 150 mg three times daily. His Crohn's symptoms have alleviated completely, and his CDAI is about 45 as a result of 3-4 diarrheas a day, possibly secondary to no longer having an ileal-cecal valve. He has had no CD flares or associated surgeries since starting bupropion. Under his doctor's supervision, he has recently tapered his azathioprine from 100 to 50 mg every day without any increase in CDAI.

These and other cases might provide impetus for prospective randomized trials to demonstrate whether or not bupropion is safe and effective for CD. If so, then bupropion might be considered for other TNF-associated diseases. Also, for patients with CD, treating physicians might need to consider “TNF implications” of all a patient's medicines.

### EXAMPLE 2

### Rheumatoid Arthritis

### EXAMPLE 3

### Multiple Sclerosis

The inflammatory Cytokine Tumor Necrosis Factor-(TNF-) has been implicated as a mediator of oligodendrocyte (OL) cell injury. TNF-is detectable within MS lesions and induces apoptosis of mature human OLs in vitro. One possible mechanism by which TNF-α mediates cell death is through the activation of c-jun N-terminal kinase (JNK). It has been shown that treatment of human OLs with TNF-α leads to activation of JNK. p53, a regulator of the cell cycle and apoptosis, is a mediator of TNF-α-induced apoptosis of OLs. In adult human OLs, p53 levels increased within 24 h after TNF-α treatment (100 ng/ml). The induced p53 was immunolocalized to the nucleus prior to the appearance of significant numbers of apoptotic cells. Overexpression of p53 by Adenovirus-mediated gene transfer into human OLs in vitro resulted in marked apoptosis as revealed by in situ cleavage of DNA (TUNEL positive), decreased Mitochondrial function, and release of Lactate Dehydrogenase into the culture medium. These in vitro studies demonstrated that increased p53 levels are associated with Apoptosis of human OLs. The findings further implicated p53 as a target for the JNK pathway activated during TNF-α-mediated cell death of human adult OLs. Clearly, Buproprion should be considered in these cases.

### EXAMPLE 3

### Glaucoma

About three million adult Americans suffer from glaucoma, making it one of the leading causes of blindness. Chronic glaucoma accounts for 90% of cases in the United States, usually appears in middle age and seems to have a genetic component. One out of five sufferers has a close relative with the condition. Doctors often refer to chronic glaucoma as the “sneak thief in the night” because it comes on gradually to steal one's vision. It may be well established before you notice the warning signs, e.g., the need for new glasses, loss of side vision, blank spots in your vision.

Other forms of glaucoma are less common but not less serious. Sudden, severe pain in the eyes, blurred vision and dilated pupils, sometimes with nausea or vomiting, may be an attack of acute, or narrow-angle, glaucoma. This type accounts for less than 10% of reported cases, but it comes on quickly and requires urgent medical attention. If left untreated, it can irreversibly damage the optic nerve, which carries visual images from the eye to the brain, causing blindness, sometimes in a matter of days.

Secondary glaucoma is usually associated with another eye disease or disorder, such as an enlarged cataract, uveitis (an inner-eye inflammation), an eye tumor or an eye injury. People suffering from diabetes are also susceptible to neovascular glaucoma, a particularly severe form of the disease. Congenital glaucoma is an extremely rare condition, affecting babies.

The eye's lens, iris and cornea are continuously bathed and nourished by a water-based fluid called aqueous humor. As new fluid is produced by cells inside the eye, excess fluid normally drains out through a complex network of tissue called the drainage angle, where the cornea and iris meet. An imbalance between the rate of production of aqueous humor and the rate of drainage will bring on chronic, or open-angle, glaucoma. This is the most prevalent form of the ailment and generally develops slowly with age.

Some people apparently inherit a condition of the inner eye in which the iris can block normal drainage channels. When this happens, the fluid does not drain out of the eye fast enough, and the sudden pressure from fluid buildup causes acute glaucoma. In newborns, defects in the drainage angle are the cause of congenital glaucoma. Both conditions need prompt medical attention to prevent potential loss of sight.

Raised intraocular pressure is one predisposing factor, but a significant portion of patients with glaucoma have normal intraocular pressure. In patients with glaucoma, and especially those with normal pressure glaucoma (NPG), it has been found that levels of the pro-inflammatory cytokine tumor necrosis factor-alpha (TNF) are elevated. Also, one gene which is mutated in autosomal dominant primary open angle glaucoma, in patients predominantly with normal or only moderately elevated intraocular pressure, seems to be associated with the TNF signaling pathway and pathogenic mutations may predispose patients to TNF associated apoptosis (Rezaie T, Child A, Hitchings R, et al. Adult-onset primary open-angle glaucoma caused by mutations in optineurin. Science 2002; 295: 1077-1079). Thus, a drug that could lower TNF may be beneficial in NPG, and we suggest that bupropion might be such a drug.

### Congestive Heart Disease

Congestive heart failure is a disease of epidemic proportions with 4.6 million patients in the U.S. and in nearly half of these patients, the etiology of their disease is unknown. It is generally viewed as a progressive disease in which initial myocardial damage is followed by cardiac remodeling and progressive dilation of the left ventricle. Cardiac compensation is followed by progressive de-compensation and patients present with worsening symptoms including fatigue, shortness of breath, and edema. Recent investigation has focused on the role of the proinflammatory cytokine TNF-in the development of heart failure and in particular in the transition from compensated to decompensated heart failure. Although TNF-can modulate the function of a group of potentially important cardiac proteins, recent evidence suggests that TNF-modulates the expression of the two families of proteins that regulate the homeostatic balance within the extracellular matrix, the matrix metalloproteinases (MMPs) and the tissue inhibitors of metalloproteinases (TIMPs). Furthermore, transgenic mice harboring cardiac restricted overexpression of TNF-develop matrix remodeling, progressive fibrosis, and collagen denaturation that are associated with up-regulation of MMP activity. Anti-TNF-therapy using adenovirus-mediated gene therapy significantly inhibited MMP activity, prevented myocardial fibrosis and collagen denaturation. These preliminary studies led us to hypothesize that the development of end-stage heart failure is due in large part to cytokine-induced matrix remodeling and fibrosis, and that the development of fibrosis marks the irreversibility of the disease.

Inventors have recently seen three patients with congestive heart failure (CHF) whose TNF-α dropped profoundly on bupropion.

A 68 year old woman with NYSHA Class I CHF who started on bupropion for major depression. Her TNF before bupropion was 347 pg/m and 5 pg/ml after two months on bupropion 150 mg BID. Her depression is partially controlled on bupropion.

A 40 year old gentleman with a history of two myocardial infarctions and NYSHA Class II CHF, who started on bupropion for major depression and desire to stop a 1.5 container/day chewing tobacco habit. His TNF before starting bupropion was 433 pg/ml, and 9 pg/ml following up titration and then four weeks on bupropion 150 mg TID). He has stopped using tobacco and his depression is in remission.

A 51 year old gentleman with NYSHA Class I CHF-TNF was 303 before bupropion was started for depression and 7 on 150 mg TID. This low TNF has been maintained for more than a year on bupropion.

While the present invention has now been described in terms of certain preferred embodiments, and exemplified with respect thereto, one skilled in the art will readily appreciate that various modifications, changes, omissions and substitutions may be made without departing from the spirit thereof. It is intended, therefore, that the present invention be limited solely by the scope of the following claims.

### REFERENCES

Altschuler E L. Monoamine oxidase inhibitors for IgA nephropathy? Medical Hypotheses 20001; 56: 225-226.

Altschuler E L. Monoamime oxidase inhibitors in rheumatoid arthritis-anti-tumor necrosis factor? Int J Immunopharm 2000; 22: 1007-1008.

Altschuler E L. Phenelzine as adjuvant treatment for Behçet's disease? International Journal of Dermatology 1999 38: 955.

Best W R, Becktel J M, Singleton J W. Rederived values of the eight co-efficients of the Crohn's Disease Activity Index (CDAI). Gastroenterology 1979;77:843-846.

Brynskov J, Nielsen O H, Ahnfelt-Ronne I, Bendtzen K. Cytokines and their natural regulation in inflammatory bowel disease: A review. Dig Dis 1994; 12: 290-304.

Farrell R J, Ang Y. Kileen P, O'Briain D S, Kelleher D, Keeling P W, Weir D G. Increased incidence of non-Hodgkin's lymphoma in inflammatory bowel disease patients on immunosuppressive therapy but overall risk is low. Gut 2000; 47: 514-519.

Garrrcha G, Smokcum R W, Stephenson J D, et al. Effect of some atypical antidepressants on-adrenoreceptor binding and adenylate cyclase activity in the rat forebrain. Eur J Pharmacol 1985;108:1-7.

Goebel M U, Mills P J, Irwin M R, Ziegler M G. Interleukin-6 and tumor necrosis factor-alpha production after acute psychological stress, exercise, and infused isoproterenol: differential effects and pathways. Psychosom Med 2000;62:591-598.

Guirao X, Kumar A, Katz J, Smith M, Lin E, Keogh C. Calvano S E, Lowry S F. Catecholamines increase monocyte TNF receptors and inhibit TNF through [symbol] -2 adrenoreceptor activation. Am J Physiol 1997; 273:E1203-1208.

Hanauer S B. Inflammatory bowel disease. N Engl J Med 1996; 334: 841-848.

Kast R E. Crohn's disease remission with phenelzine. Gastroenterology 1998; 115: 1034-1035.

Kast R E. TNF has positive and negative self-regulator feed back cycles centered around cAMP. Int J Immunopharm 2000; 22: 1001-1006.

Kast R E. Crohn's disease remission with phenelzine. Gastroenterology 1998;115:1034-1035.

Ladiwala U, Li H, Antel J P, Nalbantoglu J, Oligodendrocytes (OLs) and their Myelin membranes are the primary targets in the AutoImmune Disease Multiple Sclerosis (MS), J Neurochem 1999 August;73(2):605-11.

Leib J. Remission of rheumatoid arthritis and other disorders of immunity in patients taking monoamine oxidase inhibitors. Int J Immunopharm 1983; 5: 353-357.

Lemann M, Zenjari T, Bouhnik Y. Cosnes J, Mesnard B, Rambaud J C, Modigliani R, Cortot A, Flolombel J F. Methotrexate in CD: Long-term efficacy and toxicity. Am J Gastroenterol 2000; 95: 1730-1734.

MacDonald T T, Monteleone G, Pender S L. Recent developments in the immunology of inflammatory bowel disease. Scand J Immunol 2000 51: 2-9.

Magro F, Vieira-Coelho M A, Fraga S, Serrao M P, Tavarela-Veloso F, Tome-Ribeiro, Soares-da-Silva P. Impaired synthesis or cellullar storage of norepinephrine, dopamine and 5-hydroxytryptamine in inflammatory bowel disease (Abstract). Am J Gastro 2000; 95: 2556.

Maini R, St Clair E W, Breedveld F, Furst D, et at. Infliximab (chimeric anti-tumour necrosis factor alpha monoclonal antibody) versus placebo in rheumatoid arthritis patients receiving concomitant methotrexate: a randomised phase III trial. Lancet 1999; 354: 1932-1939.

Mundle S D, Reza S, Ali A, Mativi Y, Shetty V, Venugopal P, Gregory S A, Raza A. Correlation of tumor necrosis factor alpha (TNF alpha) with high Caspase 3-like activity in myelodysplastic syndromes. Cancer Letters 1999; 140: 201-207.

Newman W H, Zhang L M, Lee D H, Dalton M L, Warejcka D J, Castresana M R, Leeper-Woodford S K. Release of TNF from coronary smooth muscle: activation of NF-kappa B and inhibition by elevated cAMP. J Surg Res 1998; 80: 129-135.

Nikolaus S, Raedler A, Kuhbacher T, Sfikas N, et al. and others. Mechanisms in failure of infliximab for Crohn's disease. Lancet 2000; 356: 1475-1479.

Nyman U, Mussener A, Larsson E, Lorentzen J, Klarskog L. Amelioration of collagen II induced arthritis in rats by the type IV phosphodiesterase inhibitor rolipram. Clin Exp Immunol 1997; 108: 415-419.

Papadakis K A, Targan S R. Role of cytokines in the pathogenesis of inflammatory bowel disease. Ann Rev Med 2000; 51: 289-298.

Physician's Desk Reference. (Medical Economics Company, Inc., Montvale, N.J., 2000) pp. 1301-1308.

Prabhakar U, Lipshutz D, Bartus J O, Slivjak M J, Smith E F 3^{rd}, Lee J C, Esser K M. Characterization of cAMP dependent inhibition of LPS induced TNF production by rolipram, a specific phosphodiesterase IV inhibitor. Int J Immunopharm 1994; 16: 805-816.

Present DH, Rutgeerts P, Targan S, Hanauer S B, Mayer L, van Hogezand R A, Podolsky D K, Sands B E, Braakman T, De Woody K L, Schaible T F, van Deventer S J. Infliximab for the treatment of fistulas in patients with Crohn's disease. N Engl J Med 1999; 340: 1398-1405.

Reimund J M, Witterrsheim C, Dumont S, Muller C D, Kenney J S, Baumann R, Poindron P, Duclos B. Increased production of TNF, IL-1 beta, IL-6 by morphologically normal intestinal biopsies from patients with CD. Gut 1996; 39: 684-689.

Rostoker G., Rymer J. C., Bagnard G., Petit-Phar M., Griuncelli M., Pilatte Y. Imbalances in serum proinflammatory cytokines and their soluble receptors: a putative role in the progression of idiopathic IgA nephropathy (IgAN) and Henoch-Schonlein purpura nephritis, and a potential target of immunoglobulin therapy? Clinical and Experimental Immunology 1998; 114: 468-476.

Sanchez C, Hyttel J. Comparison of the effects of antidepressants and their metabolites on reuptake of biogenic amines and on receptor binding. Cell Mol Neurobiol 1999; 19: 467-489.

Sartor R B. New therapeutic approaches to Crohn;s disease. N Engl J Med 2000;342:1664-1666.

Sartor R B. New therapeutic approaches to Crohn's disease. New England Journal of Medicine 2000; 342: 1664-1666.

Sayinalp N, Ozcebe OI, Ozdemir O, Haznedaroglu I C, Dundar S, Kirazli S. Cytokines in Behcet's disease. Journal of Rheumatology 1996; 23: 321-322.

Sommer N, Loschmann P A, Northoff G H et al. The antidepressant rolipram suppresses cytokine production and prevents autoimmune encephalitis. Nature-Med 1995; 1: 244-248.

Steinhart H. Maintenance therapy in CD. Can J Gastroenterol 2000; 14 (SupplC): 23C-28C.

Talmadge J, Scott R, Castelli P, Newman-Tarr T, Lee J. Molecular pharmacology of the [symbol] -adrenergic receptor on THP-1 cells. Int J Immunopharm 1993; 15: 219-228.

Van Deventer S J. Immunotherapy of CD. Scand J Immunol 2000; 51: 18-22.

1. A method for treating Crohn's Disease in patients without causing beta-andrenergic receptor down-regulation, comprising
administering bupropion in an effective dose to alleviate or eliminate symptoms and gut pathology, and
monitoring the patient's CDAI level wherein the dose is adjusted according to degree of alleviation and elimination of symptoms

2. The method according to claim 1, wherein the effective dose is in the range of 150 mg BID to 150 mg TID for an adult patient.

3. A method for treating TNF associated diseases without causing beta-andrenergic receptor down-regulation, comprising
administering bupropion in an effective dose to alleviate or eliminate TNF associated disease symptoms and pathology; and
monitoring the patient's response wherein the dose is adjusted according to degree of alleviation and elimination of the symptoms and pathology

4. A method for treating rheumatoid arthritis in patients without causing beta-andrenergic receptor down-regulation, comprising.
administering bupropion in an effective dose to alleviate or eliminate symptoms and pathology; and
monitoring the patient's response wherein the dose is adjusted according to degree of alleviation and elimination of symptoms.

5. A method for treating psoriasis in patients without causing beta-andrenergic receptor down-regulation, comprising.
administering bupropion in an effective dose to alleviate or eliminate symptoms and pathology, and
monitoring the patient's response wherein the dose is adjusted according to degree of alleviation and elimination of symptoms

6. A method for treating IgA nephropathy in patients without causing beta-andrenergic receptor down-regulation, comprising.
administering bupropion in an effective dose to alleviate or eliminate symptoms and pathology, and
monitoring the patient's response wherein the dose is adjusted according to degree of alleviation and elimination of symptoms

7. A method for treating anemia in patients without causing beta-andrenergic receptor down-regulation, comprising:
administering bupropion in an effective dose to alleviate or eliminate symptoms and pathology, and
monitoring the patient's response wherein the dose is adjusted according to degree of alleviation and elimination of symptoms

8. The method according to claim 1, wherein the anemia treated is related to chronic disease.

9. A method for treating myelodysplasia in patients without causing beta-andrenergic receptor down-regulation, comprising.
administering bupropion in an effective dose to alleviate or eliminate symptoms and pathology; and
monitoring the patient's response wherein the dose is adjusted according to degree of alleviation and elimination of symptoms
